(19) United States
(12) Patent Application Publication
(10) Pub. No.: US 2008/0020022 A1
(43) Pub. Date: Jan. 24, 2008

(54) CHEWABLE CO-ENZYME Q-10 CAPSULE

(76) Inventor: Ronald G. Udell, Beverly Hills, CA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/757,793

(22) Filed: Jun. 4, 2007

Related U.S. Application Data
(60) Provisional application No. 60/810,918, filed on Jun. 5, 2006.

(51) Int. Cl. A61K 8/35; A61K 9/28; A61P 43/00

(52) U.S. Cl. 424/441; 514/689

(57) ABSTRACT

The present invention is directed to compositions and methods of delivery of CoQ-10 encapsulated in a chewable soft gelatin capsule.

### CROSS-REFERENCE TO RELATED APPLICATION(S)

This application claims the benefit of U.S. Provisional Application No. 60/810,918, filed Jun. 5, 2006, entitled “Chewable Co-Enzyme Q-10 Capsule” by Michael Fantuzzi, the entire contents of which is incorporated herein by reference.

### FIELD OF THE INVENTION

The present invention relates to delivery of coenzyme Q-10 and analogs thereof in a fill material encapsulated within a chewable soft gelatin capsule.

### BACKGROUND OF THE INVENTION

CoQ-10 (coenzyme Q10) is a fat-soluble quinone that is structurally similar to vitamin K and commonly known as ubiquinone. CoQ-10 is found in most living organisms, and is essential for the production of cellular energy. CoQ-10 (2,3 dimethyl-5 methyl-6-decaprenyl benzoquinone) is an endogenous antioxidant found in small amounts in meats and seafood. Although CoQ-10 is found in all human cells, the highest concentrations of CoQ-10 occur in the heart, liver, kidneys, and pancreas. It is found naturally in the organs of many mammalian species.

CoQ-10 can be synthesized in the body or it can be derived from dietary sources. Situations may arise, however, when the need for CoQ-10 surpasses the body's ability to synthesize it. CoQ-10 can be absorbed by oral supplementation as evidenced by significant increases in serum CoQ- 10 levels after supplementation.

CoQ-10 is an important nutrient because it lies within the membrane of a cell organelle called the mitochondria. Mitochondria are known as the “power house” of the cell because of their ability to produce cellular energy, or ATP, by shuttling protons derived from nutrient breakdown through the process of aerobic (oxygen) metabolism. CoQ-10 also has a secondary role as an antioxidant. CoQ-10, due to the involvement in ATP synthesis, affects the function of almost all cells in the body, making it essential for the health of all human tissues and organs. CoQ-10 particularly effects the cells that are the most metabolically active: heart, immune system, gingiva, and gastric mucosa

Several clinical trials have shown CoQ-10 to be effective in supporting blood pressure and cholesterol levels. Furthermore, CoQ-10 has also been shown to improve cardiovascular health. CoQ-10 has been implicated as being an essential component in thwarting various diseases such as certain types of cancers. These facts lead many to believe that CoQ-10 supplementation is vital to an individual's well being.

Likewise, mammals, such as pets, generally benefit from antioxidants that work together, in a variety of ways to help protect and insure health. Antioxidants neutralize harmful agents referred to as free radicals. Free radicals are formed each time a mammal takes a breath. Exposure to the suns ultraviolet rays, environmental toxins, pollution, heavy metals, stress, diet and drugs, including antibiotics, also contribute to their production. Free radicals can damage the mammalian cells and can also adversely effect vital tissues, organs and even DNA. These harmful changes accumulate and have been proven to decrease the quality and length of life. Antioxidant supplements can increase the mammal's healthy life span and slow the aging process by providing the body with additional defenses against free radicals and decreasing the resultant levels of oxidative damage. Oxidative damage has been associated with many of the leading age related degenerative diseases including cancer, heart disease, liver and kidney disorders as well as arthritis, diabetes, senility, cognitive dysfunction and canine Alzheimer's disease.

Sometimes the individual or mammal has a difficult time swallowing traditional pills due to age, gag reflex, diminished throat muscle strength due to stroke, and other ailments. Sometimes the pill is not palatable to the subject due to size, taste, or other factors.

Therefore, there is a need in the art for an improved methodology to deliver CoQ-10 to an individual or mammal, such as a dog, in need thereof.

### BRIEF SUMMARY OF THE INVENTION

The present invention pertains to chewable soft gelatin capsules containing coenzyme Q-10 within a fill material. The chewable soft gelatin shell includes at least gelatin, a plasticizer other than xylitol, xylitol and water. The fill material encapsulated within the chewable soft gelatin shell, includes coenzyme Q-10 and an acceptable carrier. The coenzyme Q-10 is selected from the group consisting of coenzyme Q-10, reduced coenzyme Q-10 and semi-reduced coenzyme Q-10.

In one embodiment the chewable soft gelatin capsule consists essentially of gelatin, a plasticizer other than xylitol, i.e., glycerin, xylitol and water.

In another embodiment, the chewable soft gelatin shell does not include a starch acetate.

In still another embodiment, the chewable soft gelatin shell does not include two types of gelatin.

In one aspect, the shell can further include maltitol.

In another aspect, the shell and/or fill material can include a coloring agent and/or a flavoring agent.

In still another aspect, the present invention also includes packaged formulations of the soft gelatin chewable capsules.

The present invention provides the advantage of a chewable soft gelatin capsule for individuals who might not otherwise easily take a pill or tablet. This is especially true for the elderly and small children where swallowing can be problematic. Likewise, animals, such as dogs, cats and horses, often do not readily take “hard” pills and tablets. The present invention solves these issues by use of a chewable capsule while delivering the beneficial coenzyme Q-10 to the subject.

In one aspect, the shell is hydrogenated starch hydrolysate free. This is advantageous for those subjects that are allergic to starch based products.

In another aspect, the shell contains less than 4% by weight of hydrogenated starch hydrolysate. Again, utilization of a very minimal amount of a hydrogenated starch hydrolysate is advantageous for those subjects that are allergic to starch based products.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### DETAILED DESCRIPTION

The present invention provides chewable soft gelatin capsules that contain an encapsulated fill material of a suitable carrier and coenzyme Q-10. In one aspect, the chewable soft gelatin shell includes at least gelatin, a plasticizer other than xylitol, xylitol and water.

The present invention also pertains to the surprising discovery that ubiquinone (CoQ-10) can be readily dissolved in varying concentrations in monoterpenes as one specific type of carrier. This forms a fill material that is encapsulated within a chewable soft gelatin shell. CoQ-10 is found in most living organisms, and is essential for the production of cellular energy. Ubiquinone is a naturally occurring hydrogen carrier in the respiratory chain (coenzyme Q) and structurally, it is a 2,3-dimethoxy-5-methyl-1,4-benzoquinone with a multiprenyl side chain, the number of isoprene units varying depending upon the organism from which it is derived. CoQ-10 analogs include reduced and semi-reduced CoQ-10 and ubiquinone derivatives described, for example, in WO 8803015, the teachings of which are incorporated herein by reference.

Generally, until the present discovery, most CoQ-10 liquid delivery methods could solubilize only up at most about 10% by weight of the CoQ-10 in the “solvent. Typical solvents included oils or the CoQ-10 was held in an aqueous suspension. Alternatively, the CoQ-10 was provided as a solid in a tablet or powder, which as discussed above, can provide difficulties for certain individuals and animals.

The present invention provides the ability to solubilize CoQ-10 in monoterpenes, as defined herein, in concentrations of up to about 60% (weight to weight) without the need to heat the solution. In one aspect, the monoterpene solubilizes CoQ-10 from about 0.1 percent by weight to about 45 percent by weight.

In particular, the solubilization of the CoQ-10 with monoterpenes can be accomplished at ambient temperatures. In one aspect, from about 5 to about 50 percent (weight CoQ-10/weight solvent) CoQ-10 can be solubilized in a monoterpene. In another aspect, from about 15 to about 40 percent w/w can be solubilized and in still another aspect, from about 20 to about 35 percent w/w CoQ-10 can be solubilized in a monoterpene.

The phrase “sufficient quantity of a monoterpene suitable to solubilize coenzyme Q-10” is therefore intended to mean that that amount of a monoterpene that will dissolve CoQ-10 under a given set of conditions, generally, those at ambient temperature. This determination should be understood by one skilled in the art and can be determined by methods known in the art, such as by solubility studies.

One of the particular advantages of utilizing monoterpenes in combination with CoQ-10 is that the enzyme is dissolved by the monoterpene. That is, many formulations currently in the marketplace have CoQ-10 present as a suspension; a situation where not all the CoQ-10 is dissolved. This reduces efficacy and the bioavailability of the CoQ-10. In one aspect, the present invention eliminates this disadvantage by solubilizing the CoQ-10 in the monoterpene.

A particular advantage in using monoterpenes is that the CoQ-10 does not have to be heated to dissolve into solution. This is important so that the CoQ-10 does not degrade upon dissolution.

The term “monoterpene” as used herein, refers to a compound having a 10-carbon skeleton with non-linear branches. A monoterpene refers to a compound with two isoprene units connected in a head-to-end manner. The term “monoterpene” is also intended to include “monoterpenoid”, which refers to a monoterpene-like substance and may be used loosely herein to refer collectively to monoterpenoid derivatives as well as monoterpenoid analogs. Monoterpenoids can therefore include monoterpenes, alcohols, ketones, aldehydes, ethers, acids, hydrocarbons without an oxygen functional group, and so forth.

It is common practice to refer to certain phenolic compounds, such as eugenol, thymol and carvacrol, as monoterpenoids because their function is essentially the same as a monoterpenoid. However, these compounds are not technically “monoterpenoids” (or “monoterpenes”) because they are not synthesized by the same isoprene biosynthesis pathway, but rather by production of phenols from tyrosine. However, common practice will be followed herein. Suitable examples of monoterpenes include, but are not limited to, limonene, pinene, cintronellol, terpinene, nerol, menthane, carveol, S-linalool, safrol, cinnamic acid, apiol, geraniol, thymol, citral, carvone, camphor, etc. and derivatives thereof. For information about the structure and synthesis of terpenes, including terpenes of the invention, see Kirk-Othmer Encyclopedia of Chemical Technology, Mark, et al., eds., 22:709-762 3d Ed (1983), the teachings of which are incorporated herein in their entirety.

In particular, suitable limonene derivatives include perillyl alcohol, perillic acid, cis-dihydroperillic acid, trans-dihydroperillic acid, methyl esters of perillic acid, methyl esters of dihydroperillic acid, limonene-2-diol, uroterpenol, and combinations thereof.

Other suitable carriers useful in the fill material include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

It should be understood that the term “comprising” (or comprises) includes the more restrictive terms consisting of and consisting essentially of.

The term “mammal” is recognized in the art and includes any of various warm-blooded vertebrate animals of the class Mammalia, including humans, characterized by a covering of hair on the skin and, in the female, milk-producing mammary glands for nourishing the young. Examples include horses, dogs, cats, cows, pigs, sheep, goats, and the like.

The term “animal” is also recognized in the art and pertains to those mammals other than humans. Examples are those above, including horses, dogs, cats, cows, pigs, sheep, goats, and the like.

The term “canine” is recognized in the art and generally refers to dogs.

The chewable soft gelatin formulations of the invention are considered dietary supplements useful to provide increased the amounts of CoQ-10 in the individuals or animals in need thereof.

Alternatively, the formulations of the invention are also considered to be nutraceuticals. The term “nutraceutical” is recognized in the art and is intended to describe specific chemical compounds found in foods that may prevent disease. CoQ-10 is one such compound.

The formulations, the fill materials, of the invention can further include various ingredients to help stabilize, or help promote the bioavailability of the CoQ-10, or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, phospholipids, L-carnitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

L-carnitine is recognized in the art and facilitates transport of materials through the mitochondrial membrane. L-carnitine is an essential fatty acid metabolism cofactor that helps to move fatty acids to the mitochondria from the cytoplasm. This is an important factor as this is where CoQ-10 uptake occurs.

In one aspect of the present invention, L-carnitine is included in soft gel formulations in combination with CoQ-10. Suitable ratios of L-carnitine and CoQ-10 are known in the art and include those described in U.S. Pat. No. 4,599,232, issued to Sigma Tau Industrie Faramaceutiche Riunite S.p.A. on Jul. 8, 1986, the teachings of which are incorporated herein in their entirety. In particular, combinations of limonene, CoQ-10 and L-carnitine in soft gel formulations are of importance. The present invention provides the advantage of solvating large amounts (relative to that of current state of the art) of CoQ-10 in limonene in a soft gel capsule along with an additive, such as L-carnitine.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, glutathione, alpha-lipoic acid, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Coloring agents useful in the fill material are generally considered oil-soluble so as to disperse/dissolve appropriately in the optional carrier if present. Suitable coloring agents include cochineal, annatto, caramel, carmine oil, carotenoids, xanthins, cryptoxanthin, paprika, carrot oils, chlorophyllin, carob extract, tumeric power/oil, anthocyanins, lycopene, astaxanthin, etc. and can be formulated in the mixture from about zero weight percent to about 5 weight percent, more specifically between about 0.5 and about 3 weight percent and in particular about 2 weight percent.

Flavoring agents useful in the fill material include cinnamon, cinnamon oil, citric acid, lemon oil, orange oil, nutmeg oil, peppermint oil, rose oil, spearmint, spearmint oil, strawberry oil, bacon flavor, barbeque flavors, beef, beef fat, cheese flavors, such as cheddar, nacho, mozzarella, romano, parmesan, chicken, clam, egg, fat, fish, ham, hot dog, lamb, lard, liver, lobster, meat and cheese blend, oyster, pizza, pork, pork liver, prawn, savory, seafood, smoked salmon, steak, taco, tallow and teriyaki and can be formulated in the mixture from about zero weight percent to about 5 weight percent, more specifically between about 0.5 and about 5 weight percent and in particular about 4 weight percent.

Chewable soft gel or chewable soft gelatin capsules can be prepared, for example, without limitation, by dispersing the formulation, as described above in an appropriate vehicle (e.g. rice bran oil, monoterpene and/or beeswax) to form a mixture. This mixture, the fill material, is then encapsulated with the chewable gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing chewable soft gelatin shells, the chewable shell can include gelatin, generally a plasticizer other than xylitol, xylitol and water. The filling of the chewable soft gelatin capsule is liquid (principally a carrier) and can include, apart from the active agent, a hydrophilic matrix. The hydrophilic matrix, if present, is a generally a polyethylene glycol having an average molecular weight of from about 200 to 1000. Alternatively, the matrix can include sorbitol and/or sorbitol special. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

Suitable plasticizers, other than xylitol, include glycerol (glycerin), sorbitol, polyglycerol, non-crystallizing solutions of sorbitol, sorbitol special, glucose, fructose and glucose syrups with different equivalents and mixtures thereof. The inclusion of glycerol provides a more chewable product.

The term “fill material” is intended to mean a substantially water-free material (generally less than about 10% water) which includes at least one active compound, CoQ-10, and optional amounts of co-solvents, buffers, surfactants, thickeners, and the like as described throughout the specification with reference to the formulations. The fill material may be of solid, semi-solid, gel, or liquid form, so long as it is compatible with the chewable soft gelatin encapsulation, so that it does not substantially degrade the chewable soft gelatin shell.

In another embodiment, the chewable soft gel capsule is prepared from gelatin, glycerin, xylitol, water and various additives. Typically, the percentage (by weight) of the gelatin is between about 15 and about 50 weight percent, in particular between about 20 and about 40 weight percent, more particularly between about 20 and about 35 weight percent, and more specifically about 35 weight percent. The formulation includes between about 10 and about 50 weight percent glycerin, in particular between about 15 and about 40 weigh percent, more particularly between about 20 and about 30 weight percent and more specifically about 31 weight percent glycerin.

A portion of the chewable capsule is typically water. The amount varies from between about 5 weight percent and about 50 weight percent, in particular between about 10 and about 40 weight percent, more particularly between about 10 and about 25 weight percent, and more specifically about 15 weight percent. The remainder of the chewable capsule components can vary, generally, between about 2 and about 25 weight percent composed of a maltitol, xylitol, flavoring agent(s), sweetener(s), coloring agent(s), etc. or combination thereof.

The amount of maltitol in the chewable soft gel shell can be varied from zero weight percent, thereby providing a hydrogenated starch hydrolysate free chewable capsule, to about 40 weight percent, more specifically between about 1 and about 4 weight percent more particularly between about 2 and about 3 weight percent.

The amount of xylitol in the chewable soft gel shell can be varied from about 1 to about 20 weight percent, more specifically from about 2 to about 10 weight percent and more particularly about 9 weight percent.

Coloring agents include those listed as being suitable for the fill material and can be formulated in the chewable soft gel shell from about zero weight percent to about 5 weight percent, more specifically between about 0.5 and about 3 weight percent and in particular about 2 weight percent.

Flavoring agents useful in the chewable soft gelatin shell include those that are generally water soluble or water dispersible and can include cinnamon, cinnamon oil, citric acid, lemon oil, orange oil, nutmeg oil, peppermint oil, rose oil, spearmint, spearmint oil, strawberry oil, bacon flavor, barbeque flavors, beef, beef fat, cheese flavors, such as cheddar, nacho, mozzarella, romano, parmesan, chicken, clam, egg, fat, fish, ham, hot dog, lamb, lard, liver, lobster, meat and cheese blend, oyster, pizza, pork, pork liver, prawn, savory, seafood, smoked salmon, steak, taco, tallow and teriyaki and can be formulated in the mixture from about zero weight percent to about 5 weight percent, more specifically between about 0.5 and about 5 weight percent and in particular about 4 weight percent.

Sweeteners include sugars and saccharin and can be present in the chewable soft gel or fill material.

After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the chewable soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a chewable soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams.

For example, the fill material for a chewable soft gel capsule containing CoQ-10 can be prepared by mixing a 35% solution of CoQ-10 and limonene (w/w) (e.g., 104 milligrams of CoQ-10 in 193.14 milligrams of limonene) with between about 0.01 grams and about 0.4 grams (e.g., 0.1 grams) tocopherol, between about 200 milligrams and about 250 milligrams (e.g., 225 milligrams) rice bran oil and between about 0.01 grams and about 0.5 grams betacarotene (e.g. about 0.02 grams). The mixture is then combined and encapsulated within a chewable soft gelatin capsule as described above.

In another embodiment, CoQ-10 (20.40 milligrams), vitamin E mixed tocopherols (53.76 milligrams), natural mixed carotenoids (4.50 milligrams), yellow beeswax (21.00 milligrams) and rice bran oil (323.327 milligrams) are mixed to prepare the fill material that provides 20 mg CoQ-10 per capsule. This fill material was used in the Examples below.

In one aspect, the chewable soft gelatin shell can be prepared by combining pork skin gelatin (200-220 bloom), glycerin (99% USP Grade), purified deionized water, D-maltitol syring (minimum 50%), xylitol, cochineal extract (AP Blend 3485), caramel liquid, titanium dioxide and bacon flavored powder.

Alternatively, the chewable soft gelatin shell can be prepared by combining pork skin gelatin, glycerin, water, agar, maltitol syrup, xylitol, cochineal extract, caramel liquid, titanium dioxide and bacon flavored powder.

The present invention also provides packaged formulations of chewable soft gelatin capsule containing the CoQ-10 fill material and instructions for its use. Typically, the packaged formulation, is administered to an individual in need thereof that requires an increase in the amount of CoQ-10 in the individual's diet. Typically, the dosage requirements is between about 1 to about 4 dosages a day.

CoQ-10 has been implicated in various biochemical pathways and is suitable for the treatment of cardiovascular conditions, such as those associated with, for example, statin drugs that effect the body's ability to product coQ-10 naturally. CoQ-10 has also been implicated in various periodontal diseases. Furthermore, CoQ-10 has been implicated in mitochondrial related diseases and disorders, such as the inability to product acetyl coenzyme A, neurological disorders, for example, such as Parkinson's disease and, Prater-Willey syndrome.

The following paragraphs enumerated consecutively from 1 through 17 provide for various aspects of the present invention. In one embodiment, in a first paragraph (1), the present invention provides a chewable soft gelatin capsule containing coenzyme Q-10, comprising a chewable soft gelatin shell that includes gelatin, a plasticizer that is not xylitol, xylitol, water, a fill material encapsulated within the chewable soft gelatin shell, comprising coenzyme Q-10 and an acceptable carrier.

2. The chewable soft gelatin capsule of paragraph 1, wherein the coenzyme Q-10 of the fill material is selected from the group consisting of coenzyme Q-10, reduced coenzyme Q-10 and semi-reduced coenzyme Q-10.

3. The chewable soft gelatin capsule of either of paragraphs 1 or 2, wherein the plasticizer of the chewable soft gelatin shell is one of glycerin, sorbitol, low molecular weight polyols or mixtures thereof.

4. The chewable soft gelatin capsule of any of paragraphs 1 through 3, wherein the plasticizer of the chewable soft gelatin shell is glycerin.

5. The chewable soft gelatin capsule of paragraphs 1 through 4, further comprising a coloring agent in the chewable soft gelatin shell.

6. The chewable soft gelatin capsule of paragraphs 1 through 5, wherein he coloring agent is carmine, caramel, titanium dioxide or mixtures thereof.

7. The chewable soft gelatin capsule of paragraphs 1 through 6, further comprising a flavoring in the fill mixture.

8. The chewable soft gelatin capsule of paragraphs 1 through 7, wherein the flavoring agent is bacon flavor.

9. The chewable soft gelatin capsule of paragraphs 1 through 8, wherein the chewable soft gelatin shell further comprises maltitol.

10. The chewable soft gelatin capsule of paragraphs 1 through 9, wherein the carrier is rice bran oil, beeswax or mixtures thereof.

11. The chewable soft gelatin capsule of paragraphs 1 through 10, wherein the chewable soft gelatin shell does not contain a hydrogenated starch hydrolysate.

13. The chewable soft gelatin capsule of paragraphs 1 through 3, wherein the maltitol in the shell is less than about 4 percent by weight of the total weight of the shell composition.

14. The chewable soft gelatin capsule of paragraphs 1 through 13, wherein the maltitol is present in an amount between about 1 and about 3 weight percent of the total weight of the shell composition.

15. The chewable soft gelatin capsule of paragraphs 1 through 14, wherein the maltitol is present in an amount of about 2 weight percent of the total weight of the shell composition.

16. A method to deliver coenzyme Q-10, comprising administering to a subject, coenzyme Q-10 encapsulated in a chewable soft gelatin capsule comprising a chewable soft gelatin shell that includes gelatin, a plasticizer that is not xylitol, xylitol, water; and a fill material encapsulated within the chewable soft gelatin shell, comprising the coenzyme Q-10 and, optionally, an acceptable carrier.

17. A packaged pharmaceutical comprising a chewable soft gelatin capsule that includes a chewable soft gelatin shell comprising gelatin, a plasticizer that is not xylitol, xylitol, water, a fill material encapsulated within the chewable soft gelatin shell, comprising coenzyme Q-10 and, optionally, an acceptable carrier and instructions of use for administration of the coenzyme Q-10.

The following examples are intended to be illustrative only and should not be considered limiting.

### EXAMPLES

Exemplary formulations of soft gelatin shell materials are as follows:

### Example 1

| Component | Weight | |
|---|---|---|
| Pork skin Gelatin 200-220 Bloom | 34.00 | kg |
| Glycerin (99%, USP Grade) | 21.90 | kg |
| Purified D.I. Water, USP | 32.75 | kg |
| D-Maltitol Syrup, minimum 50% | 10.00 | kg |
| Xylitol | 1.5 | kg |
| Cochineal Extract AP Blend 3485 | 1.00 | kg |
| Caramel liquid | 1.00 | kg |
| Titanium Dioxide | 0.15 | kg |
| Bacon Flavored Powder | 5.00 | kg |

### Example 2

| Component | Weight | |
|---|---|---|
| Pork skin Gelatin | 29.00 | kg |
| Glycerin | 20.17 | kg |
| Water | 31.50 | kg |
| Agar | 2.5 | kg |
| Maltitol Syrup | 15.00 | kg |
| Xylitol | 1.50 | kg |
| Cochineal Extract | 1.00 | kg |
| Caramel liquid | 1.00 | kg |
| Titanium dioxide | 0.15 | kg |
| Bacon Flavored Powder | 5.00 | kg |

### Example 3

| Component | Weight | |
|---|---|---|
| Gelatin | 40 | g |
| Glycerin | 35 | g |
| Purified Water | 17 | g |
| Maltitol | 3 | g |
| Xylitol | 10 | g |
| Flavoring | 5 | g |
| Coloring | 2.3 | g |

The making of this gelatin for the shell requires adding the water, maltitol, glycerin and xylitol, and heating to between about 50 and about 60° C. until the xylitol is completely melted, then adding the gelatin and heating for one hour, optionally, under vacuum between about 50 and about 60° C. To the heated mixture is added the coloring agent(s) (cochineal, caramel and titanium dioxide) into the gelatin mass (the titanium dioxide was previously mixed with an equal amount of titanium dioxide powder and glycerin, and the amount of glycerin used is represented in the total glycerin in the above formula). At this time the flavoring powder, which was previously dissolved into an equal amount of purified D.I. water (also represented in the total water in the above formula) is added and mixed into the gelatin mass, and the gelatin is placed into receivers that are heated to between about 50 and about 60° C., for between about 4 and about 6 hours.

The shell material can be used at this point to encapsulate the fill material or stored in a heated receiver to keep the shell material flowable. A suitable fill material is listed below:

| | | |
|---|---|---|
| Coenzyme Q10 | 20.4080 | mg |
| Vitamin E Mixed Tocopherols, 372 IU/g 5 | 3.7640 | mg |
| Natural Mixed Carotenoids, 20% | 4.5010 | mg |
| Yellow Beeswax | 21.0000 | mg |
| Rice Bran Oil | 320.3270 | mg |
| | 420.0000 | mg |

The resultant chewable soft gelatin capsule provides a suitable chewable capsule that can be administered to dogs to help improve the dog's well being.

Although the present invention has been described with reference to preferred embodiments, persons skilled in the art will recognize that changes may be made in form and detail without departing from the spirit and scope of the invention.

All literature and patent references cited throughout the application are incorporated by reference into the application for all purposes.

1. A chewable soft gelatin capsule containing coenzyme Q-10, comprising;
a chewable soft gelatin shell comprising:
gelatin;
a plasticizer that is not xylitol;
xylitol;
water; and
a fill material encapsulated within said chewable soft gelatin shell, comprising coenzyme Q-10 and an acceptable carrier.

2. The chewable soft gelatin capsule of claim 1, wherein said coenzyme Q-10 of said fill material is coenzyme Q-10, reduced coenzyme Q-10, semi-reduced coenzyme Q-10 or mixtures thereof.

3. The chewable soft gelatin capsule of claim 1, wherein said plasticizer of said chewable soft gelatin shell is glycerin, sorbitol, low molecular weight polyols, or mixtures thereof.

4. The chewable soft gelatin capsule of claim 1, wherein said plasticizer of said chewable soft gelatin shell is glycerin.

5. The chewable soft gelatin capsule of claim 1, further comprising a coloring agent in said chewable soft gelatin shell.

6. The chewable soft gelatin capsule of claim 5, wherein said coloring agent is carmine, caramel, titanium dioxide or mixtures thereof.

7. The chewable soft gelatin capsule of claim 1, further comprising a flavoring in said fill mixture.

8. The chewable soft gelatin capsule of claim 7, wherein said flavoring agent is bacon flavor.

9. The chewable soft gelatin capsule of claim 1, wherein said chewable soft gelatin shell further comprises maltitol.

10. The chewable soft gelatin capsule of claim 1, wherein said carrier is rice bran oil or beeswax.

11. The chewable soft gelatin capsule of claim 1, wherein said chewable soft gelatin shell does not contain a hydrogenated starch hydrolysate.

12. The chewable soft gelatin capsule of claim 9, wherein said maltitol in said shell is less than about 4 percent by weight of the total weight of the shell composition.

13. The chewable soft gelatin capsule of claim 9, wherein said maltitol is present in an amount between about 1 and about 3 weight percent of the total weight of the shell composition.

14. The chewable soft gelatin capsule of claim 9, wherein said maltitol is present in an amount of about 2 weight percent of the total weight of the shell composition.

15. A method to deliver coenzyme Q-10, comprising
administering to a subject, coenzyme Q-10 encapsulated in a chewable soft gelatin capsule comprising
a chewable soft gelatin shell comprising:
gelatin;
a plasticizer that is not xylitol;
xylitol;
water; and
a fill material encapsulated within said chewable soft gelatin shell, comprising said coenzyme Q-10 and, optionally, an acceptable carrier.

16. A packaged pharmaceutical comprising:
a chewable soft gelatin capsule comprising
a chewable soft gelatin shell comprising:
gelatin;
a plasticizer that is not xylitol;
xylitol;
water;
a fill material encapsulated within said chewable soft gelatin shell, comprising coenzyme Q-10 and, optionally, an acceptable carrier; and
instructions of use for administration of said coenzyme Q-10.
